(19) United States
(12) Patent Application Publication
(10) Pub. No.: US 2012/0149027 A1
(43) Pub. Date: Jun. 14, 2012

(54) Method for Determining the Risk of Metastasis as an Indicator for Diagnostic Imaging

(75) Inventors: Michael Untch, Berlin (DE); Ralph Markus Wirtz, Köln (DE)

(73) Assignee: SIEMENS HEALTHCARE DIAGNOSTICS INC., Tarrytown, NY (US)

(21) Appl. No.: 13/391,455

(22) PCT Filed: Jun. 29, 2010
(86) PCT No.: PCT/EP2010/003938

§ 371 Date: Feb. 21, 2012

(30) Foreign Application Priority Data
Aug. 21, 2009 (EP) 09010764

(51) Int. Cl. C12Q 1/68

(52) U.S. Cl. 435/6.12

(57) ABSTRACT

The present invention pertains to the field of in vitro diagnostics and relates to a method for determining the risk of metastasis of a tumor, wherein a high risk of metastasis shows that subsequent examinations by means of imaging methods are indicated for the patient.

The present invention is in the field of in vitro diagnostics and relates to a method for determining the metastasization risk of a tumor, wherein a high metastasization risk signifies that subsequent examinations by means of imaging methods are indicated for the patient.

Cancers are the second most common cause of death in Europe. About 100 different cancers are currently known, differing greatly in signs and symptoms, prospects of survival, and treatment options. Particularly metastasization, i.e., the dissemination of tumor cells of the primary tumor to other, in some cases far removed, organs is of crucial significance for the survival of a patient. Knowledge of the risk of a metastasization and early detection of metastases are extremely important, in order to be able to take therapeutic measures at an early stage before further organs are afflicted. In such a stage of a cancer, surgical or systemic treatment approaches have the greatest chance of success and are also more cost-effective to carry out than in later stages.

In tumor diagnostics, increasing use is made of in vitro methods for determining tumor markers, for example tissue-based genetic analyses at the RNA or DNA level or blood analyses. These markers permit prediction of the progression of a disease, efficient monitoring of the progression of the disease, and assessment of the therapy. Further applications for diagnostic tumor markers are the analysis of high-risk groups (e.g., in the case of a positive family history, liver cirrhosis, cryptorchidism, gynecological tumors), differential diagnosis of unclear tumors, for example an undetectable primary tumor, and the prognosis of the progression of a disease. The goal of tumor diagnostics is to prolong the survival of the patient affected, to improve his or her quality of life, but also to reduce treatment costs and other follow-up costs. Present genetic analyses mostly have the goal of enabling prognostic or predictive statements to be made with regard to a defined first treatment of a tumor. The statements are not specified to a particular time or to the location of metastases. The present genetic analyses also do not have the goal of predicting the failure of a primary treatment for the purposes of carrying out an adapted follow-up and second treatment.

Tumor markers are substances which are formed by the cancer itself or by the organism as a reaction to the tumor growth process. Tumor markers are present at elevated concentrations in blood or other body fluids, for example tear film, ascites, liquor or urine. Determining the concentration of tumor markers enables inferences to be made concerning the presence, the progression and the prognosis of tumors. The measurable concentration of tumor markers in body fluids is, inter alia, dependent on the total number of tumor cells (tumor mass), the reaction of the surrounding tumor stroma, the synthesis rate of the tumor marker, the blood and lymph supply to the tumor and on the marker-specific half life. However, a problem is that virtually all currently known tumor markers are also present in healthy persons at a low, but variable, concentration and not only malignant diseases can lead to elevation of tumor markers. Tumor markers which have been mostly used to date in serum and plasma diagnostics are, almost without exception, distinguished by unsatisfactory sensitivity and specificity. In particular, metastases resulting from hormone receptor-negative high-risk tumors are not detected by conventional markers such as, for example, CEA and CA15-3 [Sener Dede D, Aksoy S, Bulut N, Dizdar O, Arik Z, Gullu I, Ozisik Y, Altundag K (2009) Comparison of serum levels of CEA and CA 15-3 in triple-negative breast cancer at the time of metastases and serum levels at the time of first diagnosis. J Clin Oncol 27, 2009 (suppl; abstr e12017)], and so metastasis screening which is purely designed for tumor marker diagnostics in blood samples cannot go beyond a sensitivity of 60-70%.

The primary diagnosis of a tumor is generally diagnosed by a clinical examination, by imaging and/or endoscopic methods and/or a biopsy with subsequent pathological clarification. Subsequently, the tumor-specific laboratory parameters are determined in vitro, since tumor follow-up needs to be planned as early as at the time of primary diagnosis. Most cancers are subjected to therapy as potentially systemic diseases, since, in a still early stage, there is already a significant risk of the presence of not yet detectable micrometastases or macrometastases in distant body regions. Accordingly, systemic therapy (e.g., chemotherapy or endocrine therapy), which is aimed at controlling such still minimal residual diseases, is an integral part of the initial cancer therapy. However, a problem is that only some patients (between 10% and 70% depending on tumor type) after local treatment of the primary tumor by surgery and/or radiation therapy actually additionally have metastases in distant body regions. These minimal residual diseases are, however, not yet detectable by means of standard in vivo imaging methods, for example PET-CT, PET-MRT or MRT, at the early time of first treatment of the primary tumor. Development of nondetectable micrometastases into clinically manifest macrometastases varies in duration, depending on the tumor biology of the primary tumor, general condition of the patient, and more or less random implantation of tumor cells in distant organ systems. The current most important surrogate parameter for possible distant dissemination of primary tumors to (nonhematogenic or nonlymphogenic) organ systems, i.e., for possible metastasization, is the lymph node status of cancer patients. The lymph node status is normally determined by surgical removal of the axillary lymph nodes and pathological assessment thereof. With regard to the risk of distant metastasization, a distinction is made between patients in whom no, 1 to 3, or more than 3 lymph nodes exhibit disseminated tumor cells. This parameter alone is, however, only poorly sensitive and specific with regard to the presence of micrometastases and development thereof into lethal macrometastases and also reveals nothing about the time or site of the metastasization. In order to be able to diagnose at all the presence of micrometastases, clinical routine currently includes carrying out bone marrow biopsies, which are subsequently histopathologically examined using monoclonal antibodies to cytokeratins. The clinical relevance of these minimal diseases is, however, debatable, since the tumor cells might also be quiescent tumor cells which will never develop into distant metastases and are thus likewise to be regarded as only a further surrogate parameter for a possible, impending distant metastasization.

Since a biopsy involves an invasive procedure on the human body—often under general anesthetic—and said procedure is often associated with pain and risks for the patient, the decision to carry it out must be made carefully. If there is reasonable cause to suspect a proliferative or neoplastic change, performing a biopsy, or even direct surgery, is indicated. However, for the screening of broad asymptomatic groups, a biopsy is completely unsuitable.

Another form of tumor diagnostics is the use of what are known as imaging methods. These are understood to mean examination methods which make it possible for structures and organs of the body to be made visible. Examples thereof are examinations using X-rays, computed tomography, magnetic resonance imaging, diagnostic sonography, scintigraphy, positron emission tomography. Imaging methods also include endoscopy, which, however, requires a probe to be introduced into the body of the patient, in contrast to the abovementioned methods. Imaging methods have the disadvantage that they are associated with high costs and often involve radiation exposure. Therefore, these methods are also not suitable for screening large asymptomatic groups.

There is thus a need for an effective, preferably noninvasive method for the reliable prediction of the metastasization risk of a tumor or for the identification of cancer patients who have a high probability of developing metastases. Even if the primary tumor of a cancer patient has been successfully treated by an initial cancer therapy and the patient exhibits no more symptoms, the development of metastases remains a risk. Cancer patients for whom a high metastasization risk is diagnosed could be monitored regularly and intensively with follow-up examinations geared toward distant metastasization, preferably using imaging methods, so that metastases can be identified at a very early stage and ultimately subjected to therapy.

Intensified follow-up of all cancer patients—without prior risk stratification—did not prolong survival in previous randomized prospective studies [EBM Reviews (1994). Intensive diagnostic follow-up did not improve survival in breast cancer. ACP Journal Club 121: 77]. In addition, intensified follow-up of all cancer patients would be associated with extreme costs, since periodic imaging methods which have a high correct-negative rate (>95%) would have to be carried out. Accordingly, follow-up in asymptomatic patients which is on a regular basis and geared toward distant metastasization is currently not indicated for any cancer, as explained by the example of breast cancer [Janni W, Gerber B; Arbeitsgemeinschaft Gynäkologische Onkologie (2009). Breast Cancer Follow-Up. Gidelines Breast Version 2009.1.0. <http://www.ago-online.org/download/g_mamma_{—}09_{—}1_{—}0_{—}14 breast cancer follow up.pdf>]. Currently, a check-up by a physician on a regular basis is recommended, which is limited to palpation and to enquiring about the general state of health. Only when there is clinical suspicion, in most cases established by initial symptomatic manifestations (pain or malfunctions of distant organ systems), are adapted imaging diagnostics carried out (e.g., sonography of the liver, bone scintigram, PET-CT or MRT of the brain). Unfortunately, in these already symptomatic stages, curative treatment with the goal of healing patients is usually no longer possible, and there are high follow-up costs due to guideline-compliant, cost-intensive palliative treatments which aim at delaying death or relieving pain.

It is thus an object of the present invention to provide a noninvasive in vitro method with which the metastasization risk of a tumor can be predicted reliably, i.e., specifically and sensitively, or with which cancer patients who have a high probability of developing metastases can be identified.

The term diagnostic “sensitivity” is understood to mean the proportion of correct-positive test results as a percentage of the total number of all disease-affected persons.

The term diagnostic “specificity” is understood to mean the proportion of correct-negative test results as a percentage of the total number of non-disease-affected persons.

It was found that, surprisingly, tissue-based gene quantification, and also the combination of tissue-based gene quantification and blood-based tumor marker quantification, can predict metastasis screening with high sensitivity and meaningful specificity. This makes it possible for the first time, as early as at the time of first diagnosis or just before or after the first treatment which generally usually comprises surgery, radiation therapy, chemotherapy and also any endocrine therapies, to carry out further therapy planning which comprises an intensified follow-up program. Here, metastasis risks are predicted as a function of time, and so, in contrast to previous customary predictors, risk groups are defined which have higher occurrences of a defined metastasization event as a function of time, for example within the first three years or during years 3 to 5 or during years 8 to 12 after the first diagnosis.

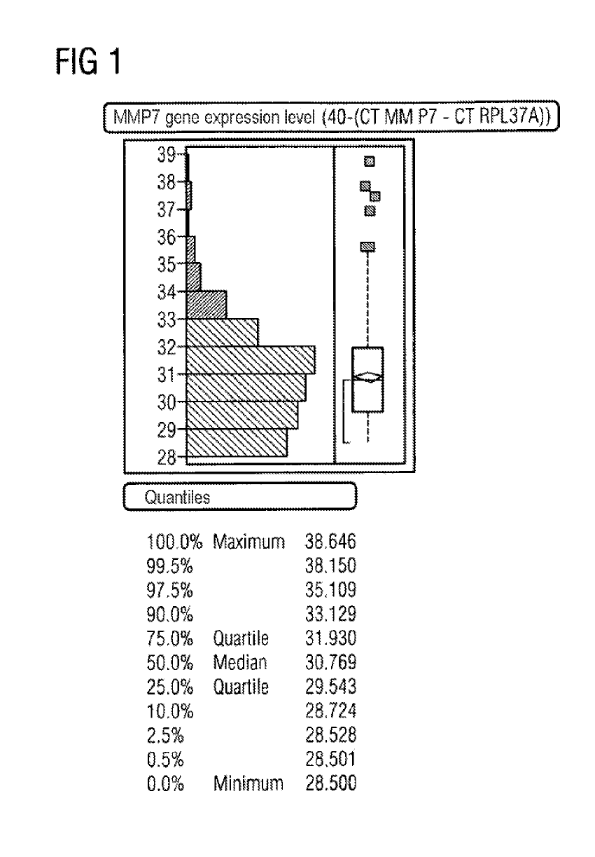

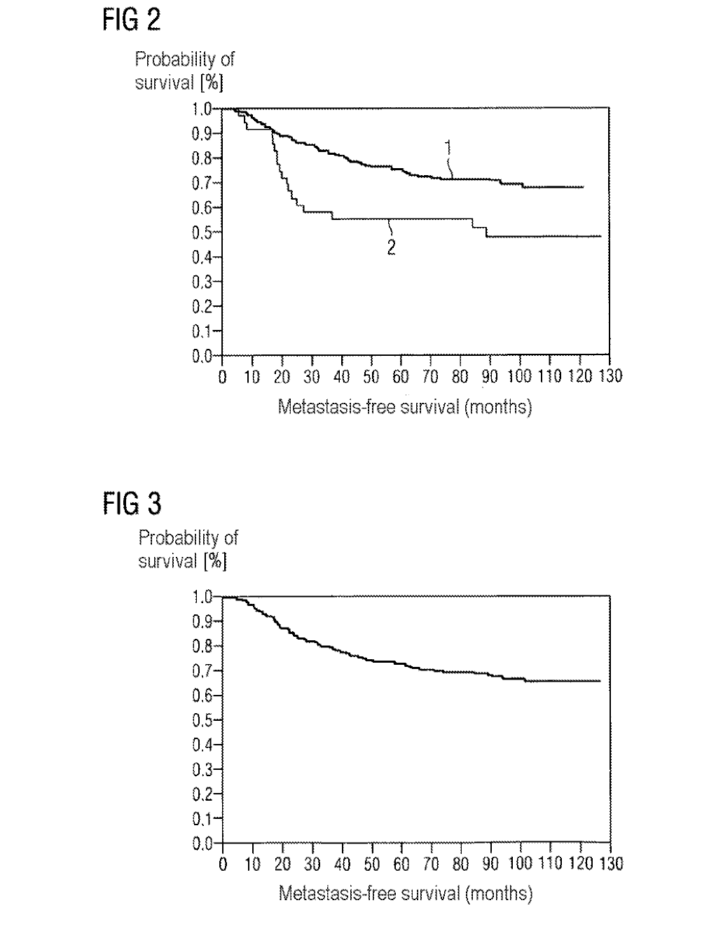

The object is solved according to the invention by quantitatively determining at least the expression of MMP7 in a biological sample from a tumor patient, wherein high (elevated) MMP7 expression denotes an increased metastasization risk, and lack of or low MMP7 expression denotes no increased metastasization risk. This was tested within the scope of the adjuvant breast cancer study HE10/97 [Fountzilas G. et al., Postoperative dose-dense sequential chemotherapy with epirubicin, followed by CMF with or without paclitaxel, in patients with high-risk operable breast cancer: a randomized phase III study conducted by the Hellenic Cooperative Oncology Group. Ann Oncol. 2005 November; 16(11): 1762-71]. Within the scope of this study, the effectiveness of two forms of therapy were tested: a first form of therapy comprising epirubicin followed by paclitaxel, followed by dose-intensified cyclophosphamide, methotrexate and fluorouracil, and a second form of therapy comprising epirubicin followed by dose-intensified cyclophosphamide, methotrexate and fluorouracil. 604 patients having advanced breast tumors (T1-3 N1 MO or T3N0M0) were randomized and treated with one of the two forms of therapy. Formalin-fixed and paraffin-embedded samples from 315 therapy-naive patients were available for analysis. RNA from a 10 μm tumor section in each case was isolated using a semiautomated, bead-based technique (Bohmann K. et al., RNA extraction from archival FFPE tissue: A comparison of manual, semi-automated and fully automated purification methods Clin Chemistry 2009, ePub Jul. 17, 2009). The relative expression levels of MMP7 were subsequently, by means of quantitative PCR using TaqMan® probes for the respective target genes and housekeeping genes, used identically to the already published data and methods [Pentheroudakis G. et al., Gene expression of estrogen receptor, progesterone receptor and microtubule-associated protein Tau in high-risk early breast cancer: a quest for molecular predictors of treatment benefit in the context of a Hellenic Cooperative Oncology Group trial. Breast Cancer Res Treat. 2009 July; 116(1): 131-43; Koutras A. K. et al., Evaluation of the prognostic and predictive value of HER family mRNA expression in high-risk early breast cancer: a Hellenic Cooperative Oncology Group (HeCOG) study. Br J. Cancer. 2008 Dec. 2; 99(11): 1775-85]. In real-time RT-PCR methodology, gene-specific fluorescent probes in which the fluorescent dye is blocked by a “quencher” are released during the real-time polymerase chain reaction (PCR) by degradation of the probes and become quantifiably detectable as light. In this methodology, the resulting fluorescence signals are proportional to the amount of the mRNA, contained in a sample, of a gene. When the released fluorescent dyes, after excitation, exceed a predefined brightness threshold (=threshold or “T”), the number of the reaction cycle is used as a measured value for the gene. The level of this measured value correlates with the amount of the mRNA, present in the tissue sample, of a target gene. The threshold is exceeded at an early time in the reaction cycles carried out that varies depending on the starting amount of the target gene. The more mRNA is present, the earlier the threshold is exceeded. The reaction cycle in which the threshold for a particular gene is exceeded is also referred to as the “CT” value (cycle of threshold). A CT value higher by a value of 1 corresponds to double the starting amount of target gene RNA, and from a mathematical point of view, this corresponds to a logarithmic scale of relative gene expression. The measured results for the target gene MMP7 were normalized to the uniformly expressed ribosomal housekeeping gene RPL37A according to the methodology published in Pentheroudakis G. et al. (2009) and in Koutras A. K. et al. (2008) (see above for full citation) and converted into DCT values (=“Delta CT” values) according to the formula “40−(CT value of MMP7-CT value of RPL37A)”. FIG. 1 shows the distribution of the RPL37A-normalized gene expression levels of MMP7 as a single gene and after normalization by the abovementioned formula. The median expression level of MMP7 after normalization to RPL37A is DCT 30.76.50% of the tumors thus have a relative expression level of MMP7 of over DCT 30.76 and therefore have elevated expression of MMP7. Furthermore, it can be seen that about 10% of the tumors have an MMP7 expression level of at least DCT 33 in FIG. 2) and therefore greatly elevated expression of MMP7. If the breast cancer patients are dichotomized into two groups on the basis of the relative expression level of MMP7 by means of the value of DCT 33, this reveals a significantly (p=0.005) shorter metastasis-free survival of the breast cancer patients intensively subjected to chemotherapy and hormone therapy (FIG. 2). About 40% of the breast cancer patients having a relative MMP7 expression level of >DCT 33 exhibit distant metastasization after just 30 months. Interestingly, there are no further metastasizations within this group after a period of more than 36 months of follow-up time.

The term “increased metastasization risk” is understood to mean a risk of metastases occurring, which risk is increased over the status-dependent baseline risk of metastases occurring for a particular tumor type taking account of the influence of any treatment, tumor size, proliferation status of the tumor cells, age of the patient, etc. For example, in the case of node-positive breast cancers, it is known that despite chemotherapy and endocrine therapy there is a baseline risk of about 15% to 20% of developing distant metastases within 5 years after the first diagnosis in the case of neoadjuvantly treated patients or after surgery in the case of adjuvantly treated patients. In the example of the adjuvant HE10/97 study, the risk of suffering from a distant metastasis within the first three years was about 20% in the tested subgroup of 315 patients. For every 5th patient of the patient group, a symptomatic distant metastasis was thus discovered within the first three years (FIG. 3). Furthermore, it is apparent that distant metastasization also becomes symptomatic beyond this period, and so ultimately about 32% of the patients exhibit distant metastasization. The diagnosis that a tumor has high MMP7 expression contains two kinds of information with regard to further follow-up. Firstly, it is indicative of an increased distant metastasization risk within the first three years, and so there is a chance, by means of imaging methods at an early stage, of identifying a metastasis early and of treating it effectively, particularly by local therapeutic measures (radiation therapy, surgery) which would no longer be possible or meaningful in further advanced stages. Secondly, it is known that in the case of patients having MMP7 tumors, if the three-year follow-up period has proceeded without distant metastasization, the likelihood of a metastasis occurring later can be virtually excluded. Accordingly, such patients no longer require a further follow-up measure. This entails not only psychological advantages for the patients but also the advantage that additional costs owing to clinical examinations lasting years can be saved. The cost savings through omitting prolonged follow-up in this patient group could thus finance in return more intensive, earlier diagnostics.

The invention provides a method for determining the metastasization risk of a tumor, wherein at least the expression of MMP7 is quantitatively determined in a biological sample from a tumor patient, wherein high (elevated) MMP7 expression denotes an increased metastasization risk and lack of or low MMP7 expression denotes no increased metastasization risk.

The method according to the invention has the advantage that an increased metastasization risk of a tumor can be reliably predicted and that, in this way, a cancer patient can be assigned to a risk group for which diagnostic follow-up examinations geared toward distant metastasization, preferably by means of imaging methods, are indicated. Furthermore, in the high-risk group for an early metastasization, a follow-up after longer than three years can be dispensed with. “Diagnostic follow-up examinations geared toward distant metastasization” are understood to mean in particular imaging methods which make it possible for structures and organs of the living body to be made visible. Examples thereof are examinations using X-rays, computed tomography (CT), magnetic resonance imaging, sonography, scintigraphy, positron emission tomography (PET), magnetic resonance tomography (MRT).

The method according to the invention permits follow-up planning for the specific further treatment of the patient as early as at the time of first diagnosis of the primary tumor or just before or after the first treatment which generally comprises surgery, radiation therapy, chemotherapy and/or an endocrine therapy. The method according to the invention thus permits the identification of patients who, owing to their increased metastasization risk, will benefit from an intensified follow-up, preferably by means of imaging methods, in that a specific examination on a regular basis by means of imaging methods makes it possible at a very early stage for metastases to be discovered and subjected to therapy, resulting in an improvement in the prospects of survival. At the same time, the method according to the invention permits the identification of patients who, owing to a nonincreased metastasization risk, would not benefit from an intensified follow-up by means of imaging methods. For these patients, the low risk makes it possible to dispense with periodic follow-up examinations geared toward distant metastasization. This has, firstly, the medical advantage that said patients are not burdened with needless examinations, for example by means of radiation-intensive imaging methods, and, secondly, the socioeconomic advantage that the costs for unnecessary examinations can be saved.

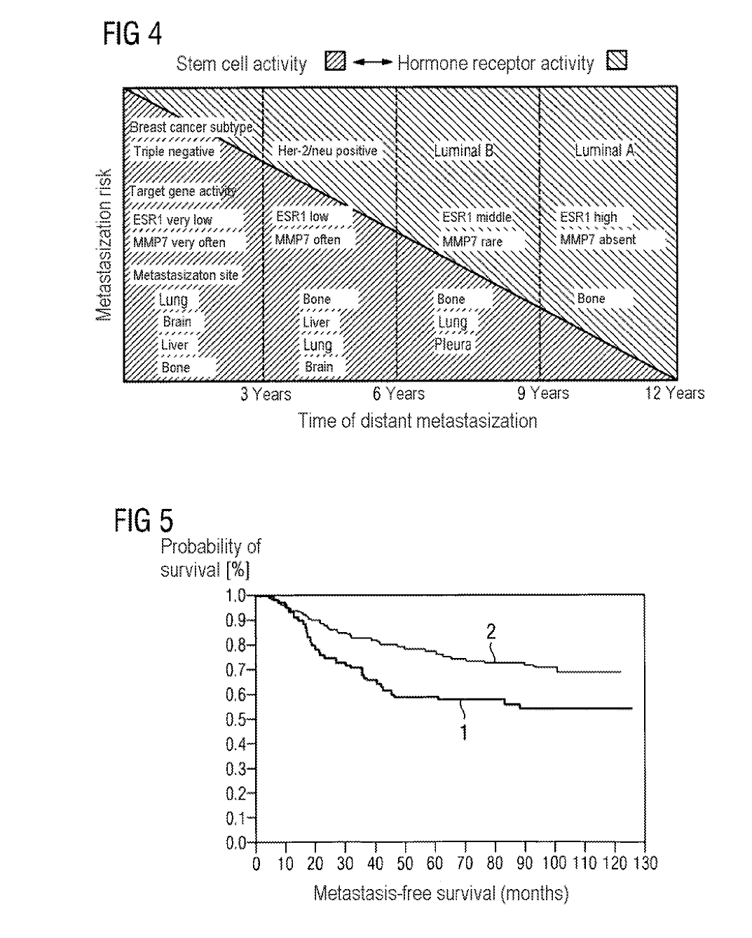

In a preferred embodiment of the method according to the invention, the expression of ESR1 is additionally quantitatively determined in a biological sample from the tumor patient, wherein lack of or low ESR1 expression denotes an increased metastasization risk and high (elevated) ESR1 expression denotes no increased metastasization risk. This determination is based on the observation that the metastasization risk can be substantially reduced to two biological motifs crucial to breast cancer, viz. stem cell activity and hormone receptor activity, in order to enable a prediction of metastasization events to be made with regard to both the time and the location (FIG. 4). Increased ESR1 gene activity correlates with bone metastasization and late metastasization events. According to the invention, the degree of direct and indirect negative interaction of stem cell activities is crucial to metastasization behavior. Estrogen receptor activity directly represses stem cell activities of transcription factors which mediate stem cell properties (e.g., Slug, Snail, FOXC2 and Twist) and indirectly inhibits other stem cell properties by upregulation of inhibitory members of stem cell activity signaling pathways (e.g., elevated expression of E-cadherin, as a result reduced WNT stem cell activity owing to the reduced amount of beta-catenin). Genes which are of interest in this connection are ESR1, MLPH, AR, ALCAM as hormone receptor marker genes and MMP7, SFRP1, Snail, Slug, FOXC2, TWIST, KRT5, notch, TGFB, OPN as stem cell activity markers. In FIG. 5, ESR1 and MMP7 are presented by way of example. For this purpose, the difference between the two negatively regulated genes MMP7 and ESR1 was determined by subtracting the non-RPL37A-normalized CT of ESR1 from the likewise non-RPL37A-normalized CT value of MMP7 (CT MMP7−CT ESR1). From a mathematical point of view, the difference can also be achieved by determining the difference of ESR1−MMP7. Both calculations are equivalent. This gene ratio is advantageous over the solution shown in FIG. 2 for various reasons. Firstly, because the ratio makes it possible for further breast cancer patients who metastasize within three years to be correctly classified as high risk. This brings about an increased sensitivity. For the MMP7 single gene detection, 36 patients are assigned to the high-risk group and reveal a distant metastasization risk of about 50% (18 out of 36 MMP7-positive patients suffer from a relapse). As can be seen in FIG. 5, for the gene ratio between MMP7 and the counterregulated ESR1, 36 patients are assigned to the high-risk group and reveal a distant metastasization risk of about 44% (35 out of 80 MMP7−ESR1-positive patients suffer from a relapse). Elevated expression of MMP7 corresponds, in the case of the difference with ESR1, to a DCT value of greater than −2.7. A further advantage of this methodology is that forming the gene ratio by subtraction of the raw CT values avoids the need to quantify one or more housekeeping genes. This reduces the risk of distorting the data owing to systematic errors, i.e., when the housekeeping gene which is by definition “neutral” and always produced uniformly is nevertheless produced not so uniformly and is produced more strongly or more weakly in certain tumors without bearing any relationship to the prognosis for the patients. This holds the danger that overestimation or underestimation of the risk might result in the case of particular tumors. As a result of the combination according to the invention of two information-bearing genes which are counterregulated, this danger no longer exists. In addition, reagent costs are thus saved, the throughput of patient samples is increased, and the option of multiplex quantification of all necessary genes in just one reaction vessel is created (e.g., one well of a microtiter plate having 384 reaction chambers).

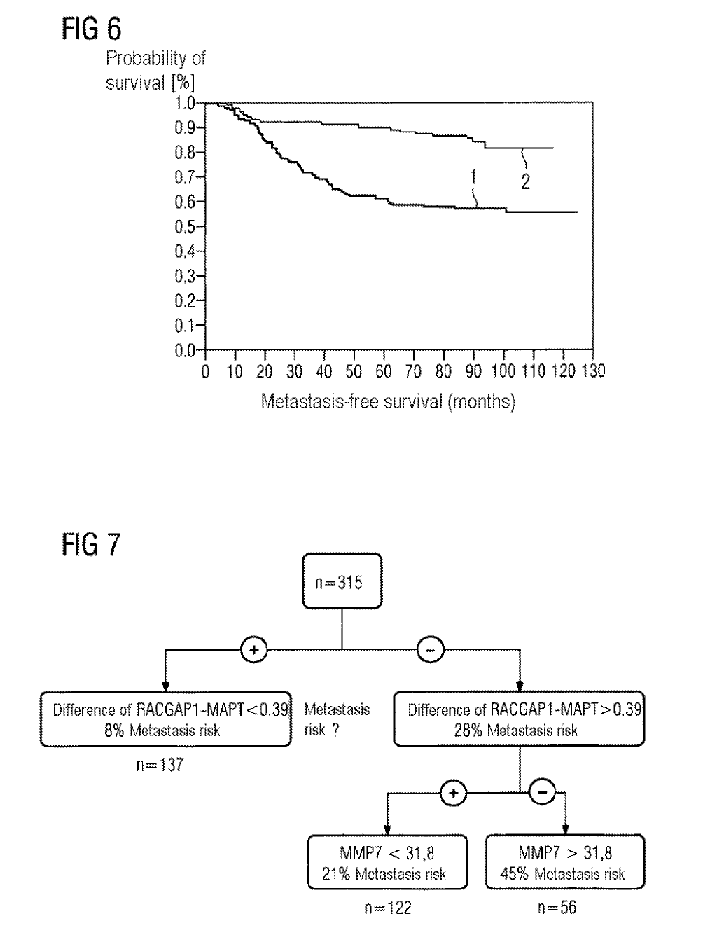

In a further preferred embodiment of the method according to the invention, the expression of MAPT and the expression of RACGAP1 are additionally quantitatively determined in a biological sample from the tumor patient, wherein low MAPT expression combined with high RACGAP1 expression denotes an increased metastasization risk and high MAPT expression combined with low RACGAP1 expression denotes a low metastasization risk (FIG. 6). It becomes apparent that the reduction to the motifs “invasion” or “cell migration” (determined by RAC GTPase-activating protein 1) and “hormone-dependent microtubule regulation” (determined by the microtubule-associated protein TAU) is also meaningful. This is particularly interesting because both proteins influence functionally the microtubule cytoskeleton and thus the change in cell morphology, as is necessary for example in the migration of cells or in the distribution of the chromosomes during cell division. Both genes or gene products contribute to opposing processes. The equilibrium between these gene products is therefore crucial to the growth behavior and migration behavior of tumor cells (FIG. 9). Both are a requirement for the process of distant metastasization. The ratio of the two processes can, for the purposes of the invention, be determined particularly by the difference between the raw CT values or else between the values normalized to the housekeeping gene RPL37A. If the this ratio is tested on the RNA extracts from the 315 primary tumors of the HE10/97 breast cancer study, there is a high distant metastasization risk for the period of the first three years for 43% of the patients (77 out of 178 RACGAP1-positive patients having a difference of RACGAP1−MAPT of >0.39 suffer from a relapse). This gene ratio is based on the rationale that both RACGAP1 and MAPT have an effect on the dynamics of the microtubule cytoskeleton, but in an opposing manner: whereas RACGAP1 brings about increased dynamics by favoring cell division and cell migration, MAPT reduces the dynamics by maintaining the microtubule structure in highly differentiated tissues. Advantageously, the number of false-negative patients in the RACGAP1-negative group thus also decreases, and so only 16% of the low-risk patients suffer from a relapse and, then, in most cases after 3 years. Since taxanes, the therapeutic goal of which is to stabilize the microtubles, were additionally administered within the scope of the HE 10/97 study investigated, the gene selection also appears reasonable from a clinical point of view.

In a further preferred embodiment, the addition of MMP7 to the expression ratio of RACGAP1 to MAPT is meaningful. For this purpose, the expression ratio of RACGAP1 to MAPT was linked to the RPL37A-normalized MMP7 gene via a decision tree having a defined cut-off (DCT of 31.8 for MMP7 and 0.39 for the RACGAP1-MAPT gene ratio) (FIG. 7). As a result, a high-risk group is identified. By adding MMP7 to the RACGAP1-MAPT ratio, it is additionally possible to distinguish a high-risk group from a medium-risk group and a low-risk group. In addition, the split because of MMP7 makes it possible to predict the metastasization site, since the MMP7-positive patient group arising as a result metastasizes preferentially in the brain, in the lungs and in the liver, more rarely in the bones, particularly when the tumors are additionally Her-2/neu-positive tumors (DCT>38 for Her-2/neu).

The method according to the invention is suitable for, inter alia, determining the metastasization risk of a tumor from the group consisting of breast cancer, ovarian cancer, colorectal cancer, lung cancer, gastric cancer, and head and neck cancer.

The expression of a gene, for example MMP7, RACGAP1, MAPT and/or ESR1, can be quantitatively determined both at the nucleic acid level (e.g., RNA hybridization techniques, RT-PCR methods, array-based methods) and at the protein level (e.g., immunological detection methods such as ELISA or RIA). Preferably, the expression of a gene is quantitatively determined by detection of mRNA by means of reverse transcription and the polymerase chain reaction (RT-PCR). The expression of a gene at the mRNA level can be quantitatively determined using any appropriate method, for example using real-time PCR or gene expression array methods, including commercially available platforms such as TaqMan®, Lightcycler®, Affymetrix, Illumina, Luminex, planar waveguides, microarray chips having optical, magnetic, electrochemical or gravimetric detection systems and others.

The expression of a gene, for example MMP7, RACGAP1, MAPT and/or ESR1, is determined in a biological sample from a tumor patient, preferably in a sample from tumor tissue which was, for example, obtained by means of a biopsy. The tumor tissue may be fresh or it may be fixed and/or embedded, for example using formalin and/or paraffin. If applicable, the biological sample must be pretreated so that the analyte(s), i.e., the gene expression products of the genes, for example proteins or mRNAs, are concentrated or made accessible for a subsequent detection reaction.

The invention will now be more particularly elucidated with reference to the accompanying examples and figures.

### EXAMPLES

The invention was tested on a breast cancer study cohort as an exemplary embodiment. Within the scope of a prospective, randomized clinical study (Hellenic Cooperative Oncology Group Trial HE10/97), 595 high-risk breast cancer patients (T1-3N1 M0 or T3N0M0) in total who were treated by chemotherapy were observed over a period of 3 years (1997-2000). Biopsies were carried out on these patients to remove tumor tissue, which was fixed with formalin and embedded in paraffin (FFPE sample material). From this FFPE sample material, mRNA was isolated by mRNA extraction and, by means of reverse transcription and associated TaqMan0 based real-time PCR (Applied Biosystems), the expression of various marker genes was quantified.

FIG. 1

shows by way of example the distribution of the MMP7 expression values for the 315 measured breast tumors from the adjuvant HE10/97 chemotherapy study. The relative expression level was determined according to the formula “40−(CT MMP7-CT RPL37A)”, and so a comparatively high numerical value corresponds to a high expression level for MMP7, whereas a low numerical value corresponds to a low MMP7 expression level. These expression values are plotted on the Y-axis, whereas the frequency of the values varying in the different measurement ranges is shown on the X-axis. The numerical values vary between 28.5 and 23.6. Owing to the logarithmic nature of the CT values of a real-time polymerase chain reaction, in which doubling of the starting amount is achieved with each reaction cycle, this corresponds to a dynamic range for marker gene expression of 2¹⁰. This means that the highest measured value is greater than the lowest value by a factor of 1024. Under the selected reaction conditions (assay design, reaction mix, PCR instrument, etc.), a value of greater than DCT 33 was defined as the threshold for elevated expression of the marker gene MMP7. The corresponding values are indicated by darker shading.

### FIG. 2

shows by way of example, using Kaplan-Meier analysis, the prognostic meaningfulness of MMP7, which in pathological routine material, i.e., in formalin-fixed resected tumors (n=315) from breast cancer patients prior to adjuvant chemotherapy who were treated within the scope of the HE10/97 study (Fountzilas et al., Ann Oncol 2005). For this purpose, the genes MMP7 and RPL37A were quantified by means of reverse transcription and associated TaqMan® based real-time PCR. The fluorescent signals produced in the real-time PCR reaction are proportional to the starting amount of the original gene products (RNAs or mRNAs) and vary, as a function of the starting amount, in when they exceed a predefined threshold (CT value). The CT values obtained in this way were then transformed into a relative and normalized gene expression level for MMP7 either by means of the formula “40-(CT target gene-CT housekeeping gene)”, i.e., “40−(CT MMP7-CT RPL37A)”. A CT value higher by a value of 1 corresponds to double the starting amount of MMP7 mRNA, and from a mathematical point of view, this corresponds to a logarithmic scale of relative gene expression. The result shows that the patients having MMP7-positive tumors (“2”; lower line) have an increased and early metastasization rate in the time frame from one year to three years. In this example, tumors defined as high expression were those whose DCT measured values, according to the formula “40−(CT MMP7-CT RPL37A)”, were above the value of 33. Patients having MMP7-negative tumors (“1”; upper line) do not have an increased and early metastasization rate in the time frame from one year to three years.

### FIG. 3

shows by way of example the distant metastasization rate in the patient group investigated. In this Kaplan-Meier diagram, the probability of survival without any distant metastasization is plotted on the Y-axis. The observation time in months is plotted on the X-axis. It can thus be seen in the breast cancer patient group investigated that about 20% of the patients exhibit distant metastases after 30 months.

### FIG. 4

shows the principle underlying the invention, that in human tumors the equilibrium between stem cell activity and hormone receptor activity is of decisive importance for both the level of the general metastasization risk (Y-axis) and for the time of distant metastasization (X-axis) and the site of distant metastasization. In addition, the breast cancer subtypes are listed according to their specific time of metastasization; for example, “triple negative” tumors (classically defined on the basis of immunohistochemical negativity for ER, PR and Her-2/neu) metastasize earlier (until three years), often exhibit elevated MMP7 expression and at the same time lowered ESR1 expression, and metastasize preferentially in the lung, brain, and liver and more rarely in bones (see also below). The reduction to two biological motifs crucial to breast cancer, i.e., stem cell activity and hormone receptor activity, enables a prediction of metastasization events to be made with regard to both the time and the location. This is based on the finding that, for example, WNT stem cell activities, measured by MMP7 marker gene expression, prevent tumor cells which have migrated into the blood circulation and/or lymphatic circulation from settling down in bones. This is particularly because of the elevated expression of SFRP1 (DCT>35 according to the formula “40−(CT SFRP1-CT RPL37A)”, i.e., after adjustment to the housekeeping gene RPL37A. SFRP1 prevents the activation of bone marrow stem cells and thus prevents circulating tumor cells from settling down in bones and development thereof into micrometastatic lesions. Tumor cells having elevated stem cell activity, shown by elevated expression of MMP7 (DCT>33), SPP1 (DCT>38), have the ability, at simultaneously low expression of SFRP1 (DCT<35), to metastasize into bones. This is particularly the case for Her-2/neu-positive tumors, which inhibit SFRP1 expression owing to Her-2/neu-induced activity.

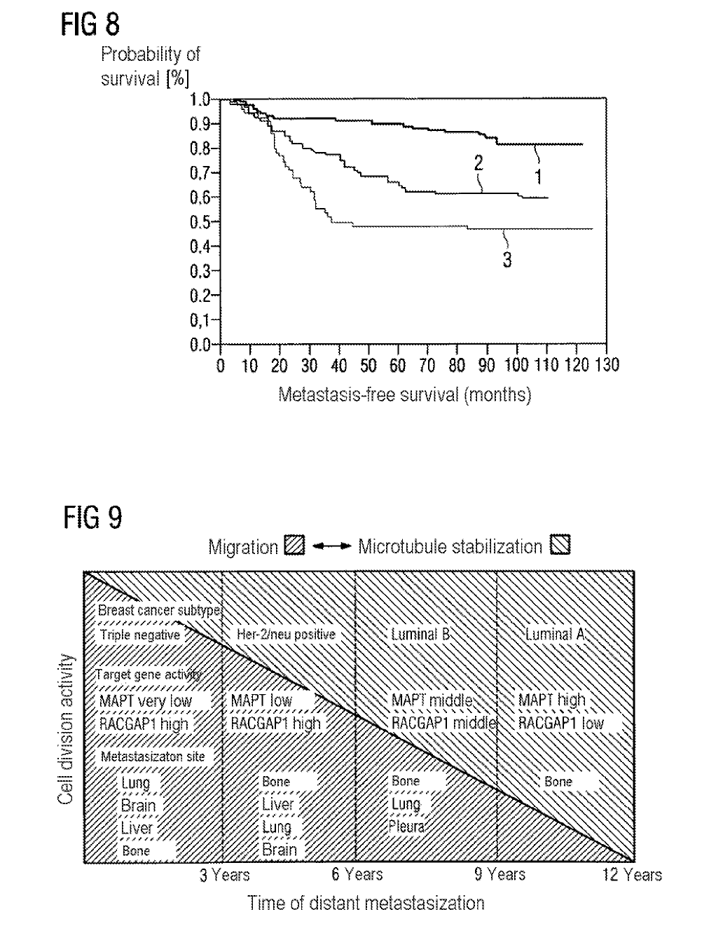

According to the invention, the degree of direct and indirect, negative interaction of stem cell activities is crucial to metastasization behavior. Estrogen receptor activity, which is substantially mediated by the isoforms of ESR1 in the case of a sufficient amount of estrogen, directly represses transcription factors which mediate stem cell properties (e.g., Slug, Snail, FOXC2 and Twist) and indirectly inhibits other stem cell properties by upregulation of inhibitory members of stem cell activity signaling pathways (e.g., elevated expression of E-cadherin, as a result reduced WNT stem cell activity owing to the reduced amount of beta-catenin). Genes which are of interest in this connection are ESR1 (elevated expression after adjustment to RPL37A at DCT 34 or above), MLPH (elevated expression after adjustment to RPL37A at DCT 34 or above), ALCAM (elevated expression after adjustment to RPL37A at DCT 34 or above), hormone receptor marker genes and MMP7 (elevated expression after adjustment to RPL37A at DCT 32 or above), SFRP1 (elevated expression after adjustment to RPL37A at DCT 35 or above), KRT5 (elevated expression after adjustment to RPL37A at DCT 34 or above), OPN (elevated expression after adjustment to RPL37A at DCT 34 or above) as stem cell activity markers. In FIG. 8, ESR1 and MMP7 are presented by way of example (see target gene activity).

### FIG. 5

shows by way of example, using Kaplan-Meier analysis, the prognostic meaningfulness of the gene ratio of MMP7 to ESR1, which was measured in formalin-fixed resected tumors (n=315) from breast cancer patients prior to adjuvant chemotherapy who were treated within the scope of the HE10/97 study (Fountzilas et al., Ann Oncol 2005). For this purpose, the genes MMP7 and ESR1 were quantified by means of reverse transcription and associated TaqMan® based real-time PCR. The result shows that the patients having an increased ratio of MMP7 to ESR1 have an increased and early metastasization rate in the time frame from one year to three years (“1”; lower red line). In this example, tumors defined as high expression were those whose DCT ratios between MMP7 and ESR1, according to the formula “(40−(CT MMP7−CT RPL37A))−(40−(CT ESR1−CT RPL37A))” or “CT MMP7−CT ESR1”, were above the value of −2.7. Patients having MMP7-negative tumors (“2”; upper green line) do not have an increased and early metastasization rate in the time frame from one year to three years over the baseline risk within this cohort shown in FIG. 3.

### FIG. 6

shows by way of example, using Kaplan-Meier analysis, the prognostic meaningfulness of the gene ratio of RACGAP1 to MAPT, which was measured in formalin-fixed resected tumors (n=315) from breast cancer patients prior to adjuvant chemotherapy who were treated within the scope of the HE10/97 study (Fountzilas et al., Ann Oncol 2005). For this purpose, the genes RACGAP1 and MAPT were quantified by means of reverse transcription and associated TaqMan® based real-time PCR. The result shows that the patients having an increased ratio of RACGAP1 to MAPT have an increased and early metastasization rate in the time frame from one year to three years (“1”; lower red line). In this example, tumors defined as high expression were those whose DCT ratios between RACGAP1 and MAPT, according to the formula “(40−(CT RACGAP1−CT RPL37A))−(40−(CT MAPT−CT RPL37A))” or “CT RACGAP1−CT MAPT”, were above the value of 0.39. Patients having RACGAP1-negative tumors (“2”; upper green line) do not have an increased and early metastasization rate in the time frame from one year to three years over the baseline risk within this cohort shown in FIG. 3.

### FIG. 7

shows by way of example a refined algorithm consisting of the gene ratio between RACGAP1 and MAPT in combination with MMP7, as a flow chart or decision tree. Primary breast tumors from 315 patients were available for the analysis. On the basis of the ratios of RACGAP1 to MAPT and the cut-off or decision point of DCT 0.39, the patients were divided into two groups. The patients having a RACGAP1-to-MAPT ratio of <0.39 are assigned to the low-risk group (“1”; 8% metastasization risk; n=137; upper red line in FIG. 8). The patients having a RACGAP1-to-MAPT ratio of >0.39 are associated with an increased risk and, on the basis of the RPL37A-normalized marker gene expression of MMP7, are assigned to a group having medium risk (“2”; n=122; 21% metastasization risk; middle green line in FIG. 8) and high risk (“3”; n=56; 45% metastasization risk; lower blue line in FIG. 8).

### FIG. 8

shows by way of example, using Kaplan-Meier analysis, the prognostic meaningfulness of the algorithm described in FIG. 7 and consisting of the gene ratio of RACGAP1 to MAPT in combination with the defined cut-off for MMP7, which were measured in formalin-fixed resected tumors (n=315) from breast cancer patients prior to adjuvant chemotherapy who were treated within the scope of the HE10/97 study (Fountzilas et al., Ann Oncol 2005). For this purpose, the genes RACGAP1, MAPT, MMP7 and RPL37A were quantified by means of reverse transcription and associated TaqMan® based real-time PCR. The result shows that the patients having an increased ratio of RACGAP1 to MAPT and non-increased MMP7 expression or increased MMP7 expression have an increased and early metastasization rate in the time frame from one year to three years (“2” and “3”; middle green line and lower blue line; 25% and 50% distant metastasization rate after three years) over the low-risk group having a low RACGAP1-to-MAPT ratio and low MMP7 expression (“1”; upper red line; 10% distant metastasization rate after three years).

### FIG. 9

shows the principle underlying the invention, that the cytoskeleton of the cell is of critical importance for cell division and cell migration. Substantially involved in this process are the microtubules, microscopically small, thread-like, intracellular connections which are crucial for the distribution of the chromosomes during cell division and for the cell shape changes which take place during cell migration. The equilibrium between migration and microtubule stabilization is of decisive importance for both the level of cell division activity (Y-axis) and for the time of distant metastasization (X-axis) and the site of distant metastasization. In addition, the breast cancer subtypes are listed according to their specific time of metastasization; for example, “triple negative” tumors (classically defined on the basis of immunohistochemical negativity for ER, PR and Her-2/neu) metastasize earlier (until three years), often exhibit elevated RACGAP1 expression and at the same time lowered MAPT expression, and metastasize preferentially in the lung, brain and liver and more rarely in bones (see also below). The reduction to two biological motifs crucial to breast cancer, which both involve regulation of the microtubule cytoskeleton, enables a prediction of metastasization events to be made. Here, it is significant that the hormone receptors ESR1 and PGR, for the purpose of cell differentiation, influence microtubule dynamics by increasing MAPT expression.

According to the invention, the degree of positive versus negative regulation of microtubule stability is crucial to metastasization behavior and resistance to chemotherapeutics. This is particularly relevant for resistance to taxane-containing therapies (such as in the HE10/97 study for example), since these are intended to elicit suicide of dividing or migrating cells by “freezing” the microtubule system. Genes which are of interest in this connection are RACGAP1 (elevated expression after adjustment to RPL37A at DCT 34 or above) and TOP2A (elevated expression after adjustment to RPL37A at DCT 34 or above) as a cell division and migration motif, and MAPT (elevated expression after adjustment to RPL37A at DCT 34 or above), ESR1 (elevated expression after adjustment to RPL37A at DCT 34 or above), PGR (elevated expression after adjustment to RPL37A at DCT 32 or above) as a microtubule stabilization and differentiation motif. In FIG. 9, RACGAP1 and MAPT are presented by way of example (see target gene activity).

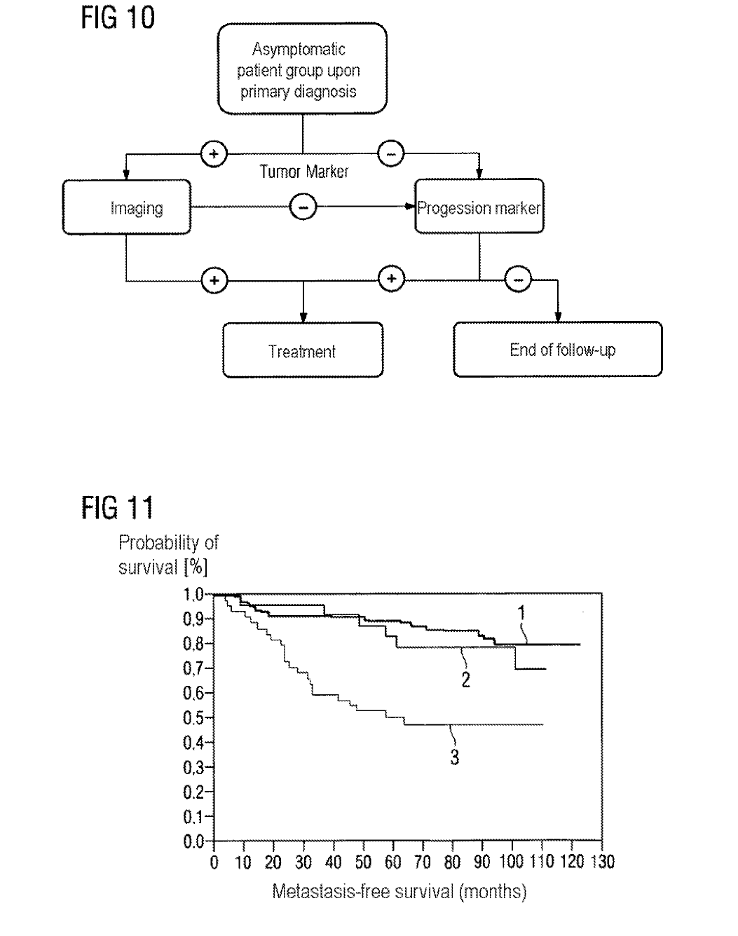

### FIG. 10

shows the method according to the invention, according to which patients for whom an increased distant metastasization risk was determined by means of tumor marker determination (in blood or in tissue) are subsequently directed to intensified follow-up (i.e., adequate imaging methods are carried out in the appropriate time frame and at the expected metastasization sites). Once an early, asymptomatic metastasis is discovered, it should be treated appropriately by means of surgical, systemic or radiological methods. False-positive patients should continue to be subjected to intensified follow-up by means of serum marker analysis or imaging. Where applicable, the follow-up can be ended after three years for the high-risk patients, since in this case no further metastasization is to be expected.

### FIG. 11

shows by way of example, using Kaplan-Meier analysis, the prognostic meaningfulness of an algorithm which, based on the decision tree from FIG. 7, has the gene ratio of RACGAP1 to MAPT as a basis, but then identifies within the tumors having an increased RACGAP1-to-MAPT ratio (DCT>0.39) by means of ALCAM those tumors which have a low metastasis risk. For this purpose, the decision point used is the cut-off of the RPL37A-normalized ALCAM values at a DCT value of 35.4. Tumors having ALCAM expression above DCT 35.4 have a lower risk (“2”; middle green line; 10% distant metastasization rate after three years) than tumors having lower ALCAM expression (“3”; lower blue line; 44% distant metastasization rate after three years). The low-risk group is defined by a low ratio of RACGAP1 to MAPT (DCT<0.39) and is identical to the low-risk group from FIGS. 8 and 6.

TABLE 1
Gene markers, sequence accession numbers, etc.

| Marker name | Accession no. | Gene name, synonyms |
|---|---|---|
| MMP7 | NM_002423.3 | Homo sapiens matrix |
| | | metallopeptidase 7 (matrilysin, |
| | | uterine) (MMP7), mRNA |
| ESR1 | NM_001122742.1 | Homo sapiens estrogen receptor 1 |
| | | (ESR1), transcript variant 4, mRNA; |
| | | estrogen receptor alpha isoform 4 |
| ESR1 | NM_001122741.1 | Homo sapiens estrogen receptor 1 |
| | | (ESR1), transcript variant 3, mRNA; |
| | | estrogen receptor alpha isoform 3 |
| ESR1 | NM_001122740.1 | Homo sapiens estrogen receptor 1 |
| | | (ESR1), transcript variant 2, mRNA; |
| | | estrogen receptor alpha isoform 2 |
| ESR1 | NM_000125.3 | Homo sapiens estrogen receptor 1 |
| | | (ESR1), transcript variant 1, mRNA; |
| | | estrogen receptor alpha isoform 1 |
| ESR1 | BX640939 | estrogen receptor alpha variant; |
| | | usage of alternative promoter in |
| | | intron 1 and alternative 3′ end |
| MAPT | NM_001123066.2 | Homo sapiens microtubule-associated |
| | | protein tau (MAPT), transcript |
| | | variant 6, mRNA; microtubule- |
| | | associated protein tau isoform 6 |
| MAPT | NM_001123067.2 | Homo sapiens microtubule-associated |
| | | protein tau (MAPT), transcript |
| | | variant 5, mRNA; microtubule- |
| | | associated protein tau isoform 5 |
| MAPT | NM_016841.3 | Homo sapiens microtubule-associated |
| | | protein tau (MAPT), transcript |
| | | variant 4, mRNA; microtubule- |
| | | associated protein tau isoform 4 |
| MAPT | NM_016834.3 | Homo sapiens microtubule-associated |
| | | protein tau (MAPT), transcript |
| | | variant 3, mRNA; microtubule- |
| | | associated protein tau isoform 3 |
| MAPT | NM_005910.4 | Homo sapiens microtubule-associated |
| | | protein tau (MAPT), transcript |
| | | variant 2, mRNA; microtubule- |
| | | associated protein tau isoform 2 |
| MAPT | NM_016835.3 | Homo sapiens microtubule-associated |
| | | protein tau (MAPT), transcript |
| | | variant 1, mRNA; microtubule- |
| | | associated protein tau isoform 1 |
| RACGAP1 | NM_013277.3 | Homo sapiens Rac GTPase activating |
| | | protein 1 (RACGAP1), transcript |
| | | variant 1, mRNA; Rac GTPase |
| | | activating protein 1 |
| RACGAP1 | NM_001126103.1 | Homo sapiens Rac GTPase activating |
| | | protein 1 (RACGAP1), transcript |
| | | variant 2, mRNA; Rac GTPase |
| | | activating protein 1 |
| RACGAP1 | NM_001126104.1 | Homo sapiens Rac GTPase activating |
| | | protein 1 (RACGAP1), transcript |
| | | variant 3, mRNA; Rac GTPase |
| | | activating protein 1 |
| MLPH | NM_001042467.1 | Homo sapiens melanophilin (MLPH), |
| | | transcript variant 2, mRNA; |
| | | melanophilin isoform 2 |
| MLPH | NM_024101.5 | Homo sapiens melanophilin (MLPH), |
| | | transcript variant 1, mRNA; |
| | | melanophilin isoform 1 |
| Her-2/neu | NM_004448.2 | Homo sapiens v-erb-b2 erythroblastic |
| | | leukemia viral oncogene homolog 2, |
| | | neuro/glioblastoma derived oncogene |
| | | homolog (avian) (ERBB2), transcript |
| | | variant 1, mRNA; erbB-2 isoform a |
| Her-2/neu | NM_001005862.1 | Homo sapiens v-erb-b2 erythroblastic |
| | | leukemia viral oncogene homolog 2, |
| | | neuro/glioblastoma derived oncogene |
| | | homolog (avian) (ERBB2), transcript |
| | | variant 2, mRNA; erbB-2 isoform b |
| PGR | NM_000926.4 | Homo sapiens progesterone receptor |
| | | (PGR), mRNA |
| RPL37A | NM_000998.4 | Homo sapiens ribosomal protein |
| | | L37a (RPL37A), mRNA |
| SPP1 | NM_001040058.1 | Homo sapiens secreted |
| | | phosphoprotein 1 (SPP1), |
| | | transcript variant 1, mRNA; |
| | | secreted phosphoprotein 1 isoform a |
| SPP1 | NM_000582.2 | Homo sapiens secreted |
| | | phosphoprotein 1 (SPP1), |
| | | transcript variant 2, mRNA; |
| | | secreted phosphoprotein 1 isoform b |
| SPP1 | NM_001040060.1 | Homo sapiens secreted |
| | | phosphoprotein 1 (SPP1), |
| | | transcript variant 3, mRNA; |
| | | secreted phosphoprotein 1 isoform c |
| SFRP1 | NM_003012.3 | secreted frizzled-related protein 1 |
| KRT 5 | NM_000424.3 | Homo sapiens keratin 5 (KRT5), |
| | | mRNA |
| ALCAM | NM_001627.2 | Homo sapiens activated leukocyte |
| | | cell adhesion molecule |
| | | (ALCAM), mRNA |
| TOP2A | NM_001067.2 | Homo sapiens topoisomerase |
| | | (DNA) II alpha 170 kDa (TOP2A), |
| | | mRNA |

The sequence accession numbers originate from the following database:

<http://genome.ucsc.edu/>

### UCSC Genome Browser:

Kent W J, Sugnet C W, Furey T S, Roskin K M, Pringle T H, Zahler A M, Haussler D. The human genome browser at UCSC. Genome Res. 2002 June; 12(6): 996-1006.
Karolchik D, Kuhn R M, Baertsch R, Barber G P, Clawson H, Diekhans M, Giardine B, Harte R A, Hinrichs A S, Hsu F, Miller W, Pedersen J S, Pohl A, Raney B J, Rhead B, Rosenbloom K R, Smith K E, Stanke M, Thakkapallayil A, Trumbower H, Wang T, Zweig A S, Haussler D, Kent W J. The UCSC Genome Browser Database: 2008 update. Nucleic Acids Res. 2008 January; 36: D773-9.

1. A method for determining the metastasization risk of a tumor, characterized in that at least the expression of MMP7 is quantitatively determined in a biological sample from a tumor patient, wherein elevated MMP7 expression denotes an increased metastasization risk, and lack of or low MMP7 expression denotes no increased metastasization risk.

2. The method as claimed in claim 1, further characterized in that the expression of ESR1 is additionally quantitatively determined in a biological sample from the tumor patient, wherein lack of or low ESR1 expression denotes an increased metastasization risk, and elevated ESR1 expression denotes no increased metastasization risk.

3. The method as claimed in claim 1, further characterized in that the expression of MAPT and the expression of RACGAP1 are additionally quantitatively determined in a biological sample from the tumor patient, wherein low MAPT expression combined with high RACGAP1 expression denotes an increased metastasization risk, and high MAPT expression combined with low RACGAP1 expression denotes no increased metastasization risk.

4. A method for determining the metastasization risk of a tumor, characterized in that at least the expression of MAPT and the expression of RACGAP1 are quantitatively determined in a biological sample from a tumor patient, wherein low MAPT expression combined with high RACGAP1 expression denotes an increased metastasization risk, and high MAPT expression combined with low RACGAP1 expression denotes no increased metastasization risk.

5. The method as claimed in claim 4, further characterized in that the expression of ALCAM is additionally quantitatively determined in a biological sample from the tumor patient, wherein low ALCAM expression denotes an increased metastasization risk, and elevated ALCAM expression denotes no increased metastasization risk.

6. The method as claimed in claim 1 for determining the metastasization risk of a tumor from the group consisting of breast cancer, ovarian cancer, colorectal cancer, lung cancer, gastric cancer, and head and neck cancer.

7. The method as claimed in claim 1, further characterized in that the expression of MMP7, ESR1, MAPT, RACGAP1 and/or ALCAM is quantitatively determined by measurement of mRNA amount.

8. An in vitro method for identifying tumor patients who have a high probability of benefiting from an examination by means of an in vivo imaging method, characterized in that the metastasization risk of the tumor of a patient is determined using a method as claimed in claim 1 and wherein an increased metastasization risk signifies that the patient has a high probability of benefiting from an examination by means of an in vivo imaging method.
